(19) United States
(12) Patent Application Publication
(10) Pub. No.: US 2016/0333318 A1
(43) Pub. Date: Nov. 17, 2016

(54) METHOD FOR THE ISOLATION, PURIFICATION AND AMPLIFICATION OF RENAL PROGENITORS CD133+CD24+ FROM THE URINE OF PATIENTS SUFFERING FROM RENAL DISEASES

(71) Applicant: AZIENDA OSPEDALIERO-UNIVERSITARIA MEYER, Firenze (IT)

(72) Inventors: Paola ROMAGNANI, Firenze (IT); Elena LAZZERI, Firenze (IT); Laura LASAGNI, Firenze (IT)

(73) Assignee: AZIENDA OSPEDALIERO-UNIVERSITARIA MEYER, Firenze (IT)

(21) Appl. No.: 15/108,082

(22) PCT Filed: Dec. 23, 2014
(86) PCT No.: PCT/IB2014/067271

§ 371 Date: Jun. 24, 2016

(30) Foreign Application Priority Data
Dec. 24, 2013 (IT) FI2013A000303

(51) Int. Cl. C12N 5/071; G01N 33/50; G01N 33/569

(52) U.S. Cl. CPC C12N 5/0687; G01N 33/56966; G01N 33/5044; G01N 33/5014; C12N 5/0686; C12N 2501/50; G01N 2333/70596; G01N 2800/347; C12N 2501/12

(57) ABSTRACT

The present invention describes a non-invasive method to isolate with high efficiency, purity and reproducibility the population of renal progenitors CD133+CD24+, from urine samples of patients suffering from various glomerular diseases. Said renal progenitors can then easily be induced to differentiate into podocytes. The isolation of renal progenitors with the method of the invention allows the use of said cells as a cellular model of a disease for the in vitro study of genetic excluding exfoliated epithelial cells and blood cells mutations due to the podocyte or for the study of renal toxicity induced by potentially nephrotoxic drugs on the tubules.

### FIELD OF THE INVENTION

The present invention relates to the field of methods for the isolation of pluripotent cells from urine samples.

### BACKGROUND ART

Recent studies have shown that, as a result of glomerular injury, glomerular epithelial cells are detached and are lost in the urine as demonstrated both in mouse models and in human glomerular diseases. Their excretion in urine is proposed as a useful non-invasive marker for assessing the activity of the glomerular disease in patients suffering from various glomerular diseases such as focal segmental glomerulosclerosis (FSGS), membranous glomerulonephritis (MGN), membrano-proliferative glomerulonephritis (MPGN) and IgA nephropathy.

Recent evidence suggests that cells isolated from the urine do not constitute a homogeneous population but, rather, are a heterogeneous population expressing both podocyte markers and markers characteristic of parietal epithelial cells of the Bowman's capsule. These results suggest, therefore, that in the course of activity of a glomerular disease, a significant number of renal progenitors, residing at the level of Bowman's capsule, can react by proliferating and detaching from their seat. The excretion in urine of renal progenitors would offer the possibility to isolate them from urine samples of patients suffering from glomerular conditions. In agreement with the hypothesis of isolating stem cells from the urine of patients, recent work has shown that stem cells can be isolated from human urine samples. These cells showed in vitro characteristics of multipotent progenitor able to differentiate into multiple cellular lineages. However, all the methods described to date have not well characterized the specific population of progenitors obtained and have purified stem or progenitor populations having low efficiency and purity. It is therefore clear that there is a need to have a non-invasive method to isolate with high efficiency, purity and reproducibility the population of renal progenitors CD133+CD24+ which may then be easily induced to differentiate into podocytes.

### DEFINITIONS AND ABBREVIATIONS

PBS=PHOSPHATE-BUFFERED SALINE
FBS=Fetal Bovine Serum
EGM-MV=endothelial cell growth medium-microVascular
VRAD=1,25-dihydroxyvitamin D3 [1,25(OH)2D3]- and all-trans-retinoic acid
(ATRA)-supplemented differentiation medium
(VRAD)

### SUMMARY OF THE INVENTION

The present invention relates to a method for the isolation, purification and amplification of renal progenitor cells of a patient, said method comprising the following sequence of operations:
subjecting a sample of urine obtained from a patient suffering from a renal disease to a first centrifugation;
removing the supernatant and re-suspending the pellets in PBS;
subjecting to a second centrifugation;
removing the supernatant and
transferring the cells on a cell culture plate and growing in a culture medium comprising EGM-MV 20% FBS and a mixture of antibiotics including penicillin, streptomycin and rifampicin;
after 5-7 days of culture, removing the culture medium, washing the culture plate with PBS and then adding said fresh culture medium;
growing for at least another 7-9 days, in said fresh culture medium and then subsequently up to confluence of a population of cells characterized by the expression of surface markers CD133 and CD24 characteristic of renal progenitors.

The cells obtained by the method of the invention are highly purified. The degree of purification with which they were obtained is much higher than what was possible with the methods known in the art.

The morphological characterization showed that the cells isolate from the urine, according to the method of the invention, are morphologically identical to those of renal progenitors CD133+CD24+ isolated from kidney tissue, therefore allowing establishing that urine represents a new source from which to isolate, in an easy, non-invasive, fast and efficient manner, a highly purified population of renal progenitors CD133+CD24+.

The availability of these cells to study the mechanisms underlying the process of regeneration of renal damage is a prospect of crucial importance for the understanding of the mechanisms that may become new targets for a possible therapeutic treatment.

The present invention therefore proposes the use of renal progenitors CD133+CD24+ for future diagnostic use as cellular model for the screening of drugs or for the study of the functional role of unknown mutations involved in renal diseases.

The object of the present invention is also a diagnostic method which comprises the isolation of renal progenitors from the urine of patients suffering from a renal disease, either glomerular or tubular, and more particularly genetic, said isolation according to the method of the invention.

A further object of the invention is the use of renal progenitors isolated according to the method of the invention, from the urine of patients suffering from a genetic glomerular or tubular disease, such as cellular models for the in vitro screening of drugs for the treatment of said disease or for the in vitro study of the functional role of unknown mutations involved in renal diseases.

An object of the present invention is also a diagnostic method for the patient-specific prediction of the renal toxicity of potentially nephrotoxic drugs, said method comprises the isolation of renal progenitors, according to the method of the invention, from the urine of the patient suffering from any disease which requires subjecting to treatment with potentially nephrotoxic drugs.

An object of the present invention is also a kit of parts for the simultaneous, separate or sequential use in the method according to the invention, said kit comprising at least one container containing a culture medium comprising EGM-MV 20% FBS and a mixture of antibiotics including penicillin, streptomycin and rifampicin.

### BRIEF DESCRIPTION OF THE FIGURES

FIG. 1—shows the total number of urine samples collected for implementing the method of the invention;

FIG. 2—shows the flow chart of the method of the present invention that allows the isolation of renal progenitors cells CD133+CD24+;

FIG. 3—shows A) Microscopy image showing the morphology of the cells isolated from the urine, according to the method of the invention, and expression of surface markers characteristic of renal progenitors CD133+CD24+ such as CD133, CD24 and CD106 as demonstrated by the flow cytometric analysis. B) expression of markers CD133, CD24, cytokeratin, vimentin and uroplakin III in cells isolated from the urine, according to the method of the invention, and evaluated by confocal microscopy. C) volcano plots showing the gene expression profile of GPCRs, of inflammatory genes and of miRNAs in cells isolated from the urine, according to the method of the invention, and in renal progenitors CD133+CD24+.

FIG. 4—shows A) Schematic example of the mutations identified in three patients: case FD compound heterozygous mutation in the NPHS2 gene, case CL homozygous mutation in the NPHS2 gene; case BCW heterozygous mutation in the LMX1B gene. B) Expression of nephrin on a sample of renal progenitors CD133+CD24+ obtained from patients with glomerular disease but with no genetic mutations (healthy) used as a control and on samples of renal progenitors CD133+CD24+ obtained from three patients mutated after differentiation in podocyte. C) Assessment of mRNA levels of nephrin in samples of renal progenitors CD133+CD24+ obtained from three patients mutated after differentiation in podocyte and their comparison with the control sample (WT) obtained from patients without genetic mutations. D) Expression of podocin (NPHS2) on a sample of renal progenitors CD133+CD24+ obtained from patients without genetic mutations (healthy) used as a control and on samples of renal progenitors CD133+CD24+ obtained from three patients mutated after differentiation in podocyte. E) Assessment of mRNA levels of podocin in samples of renal progenitors CD133+CD24+ obtained from three patients mutated after differentiation in podocyte and their comparison with the control sample (WT) obtained from patients without genetic mutations. F) Assessment of the cytoskeleton after staining with phalloidin (green) in all three patients analyzed by confocal microscopy. Counterstaining of the nuclei with To-pro-3.u-RPC: urine-derivedrenal progenitor cells CD133+CD24+.

FIG. 5—shows A) Assessment of mRNA levels of tubule-specific markers in samples of renal progenitors CD133+CD24+ (n=8) isolated from urine after differentiation to the tubular phenotype. B) Expression of tubular markers in cultures of renal progenitors CD133+CD24+ isolated from the urine, before (day 0) and after differentiation (day 21) to the tubular phenotype. Counterstaining of the nuclei with To-Pro-3. Bars 20 μm.C) Assessment of the percentage of dead cells on samples of renal progenitors CD133+CD24+ differentiated in tubular cells and subjected for 24 h to various doses of doxorubicin, by cytofluorimetric analysis of the staining for annexin V and propidium iodide (PI).

### DETAILED DESCRIPTION OF THE INVENTION

Preferably, the centrifugation of the method of the invention is carried out at 1200-1800 rpm, more preferably at 1400-1500 rpm, for a time ranging between 3 and 15 min. Preferably, the first centrifugation can take place at 1400 rpm for 10 minutes and the second at 1400 rpm for 5 minutes.

The culture medium of the method according to the invention comprises EGM-MV 20% FBS and a mixture of antibiotics. Said mixture of antibiotics preferably consists of penicillin, streptomycin and rifampicin. More preferably, said mixture consists of 100 U/mL penicillin, 1 mg/mL streptomycin and 8 mcg/mL rifampicin. After the first 6 days of culture, the medium is removed, the plate washed and fresh medium is added, said fresh medium still comprising EGM-MV 20% FBS and a mixture of antibiotics.

After removing the first culture medium and washing, the removal, of non-adherent cells and culture debris culture from the culture plate is obtained. The phase of culture in the presence of the antibiotic mixture is carried out for a total of about 15 days.

At the end of this phase of cell culture in the presence of antibiotic agents, a selected cell culture is obtained that is free from bacterial contamination (frequently of bacteria belonging to the group of Enterococci).

After the 15 days of culture in the presence of the mixture of antibiotics it is possible to keep the culture in the absence of antibiotics.

The next phase of culture, the renal progenitors having already been selected, and with surprisingly high purity, is a culture of amplification of said cells.

The renal progenitor cells isolated by the method of the invention preferably show absence of expression of uroplakin Ill, a marker characteristic of urothelium, demonstrating, therefore, the renal origin of the cells isolated from the urine. They preferably also express CD106, more preferably also the markers cytokeratin and vimentin.

The object of the present invention therefore also are the cells obtained by the present method which for the first time allows obtaining for each individual patient, by means of a totally non-invasive method, a population of renal progenitors specific to that disease (and in that patient).

A possible clinical application of this invention is based on the isolation of renal progenitors CD24+CD133+ from urine samples of patients, preferably pediatric, suffering from renal diseases, also genetically transmitted, such as children with steroid-resistant nephrotic syndrome associated with mutations on the podocin gene (NPHS2) and on the LMX1B gene, transcription factor that regulates the expression of many podocyte genes.

The isolation of the renal progenitors CD133+CD24+ from the urine of patients with renal diseases, specifically genetic diseases such as steroid-resistant nephrotic syndrome, finally makes it possible to obtain a cellular model for the in vitro study of the effect of known and unknown mutations at the basis of renal diseases (FIG. 4). This can allow the causal diagnosis of a genetic disease even when the mutation is unknown, using such cells as a model of disease in clinical setting.

Preferably, the method of the invention can be applied to urine samples from patients suffering from steroid-resistant nephrotic syndrome associated with genetic mutations. More preferably, said patients are children.

Preferably, the method of the invention can be applied to urine samples from patients suffering from oncological diseases which require chemotherapy treatment. More preferably, said patients are children.

The renal progenitors CD133+CD24+, obtained with the method of the invention, as described above, can then be differentiated to the podocyte phenotype. The differentiation can be carried out by growing the renal progenitor cells CD133+CD24+ in a differentiation medium (VRAD) consisting of DMEM/F12 supplemented with vitamin D3 and retinoic acid, for about 48 h.

Nephrotoxicity following chemotherapy treatments is a common phenomenon that is difficult to predict and is primarily influenced by patient-specific genetic factors making the renal tubular cells particularly susceptible to the harmful effects of drugs. Since renal progenitors can be differentiated into tubular cells and the susceptibility to the effects of nephrotoxic drugs is a genetically determined phenomenon that would require a personalized model of drug screening, we believe that the cultures of renal progenitors purified from the patient, who will be subjected to treatment with potentially nephrotoxic drugs, might represent an innovative cellular model on which to make patient-specific functional assays.

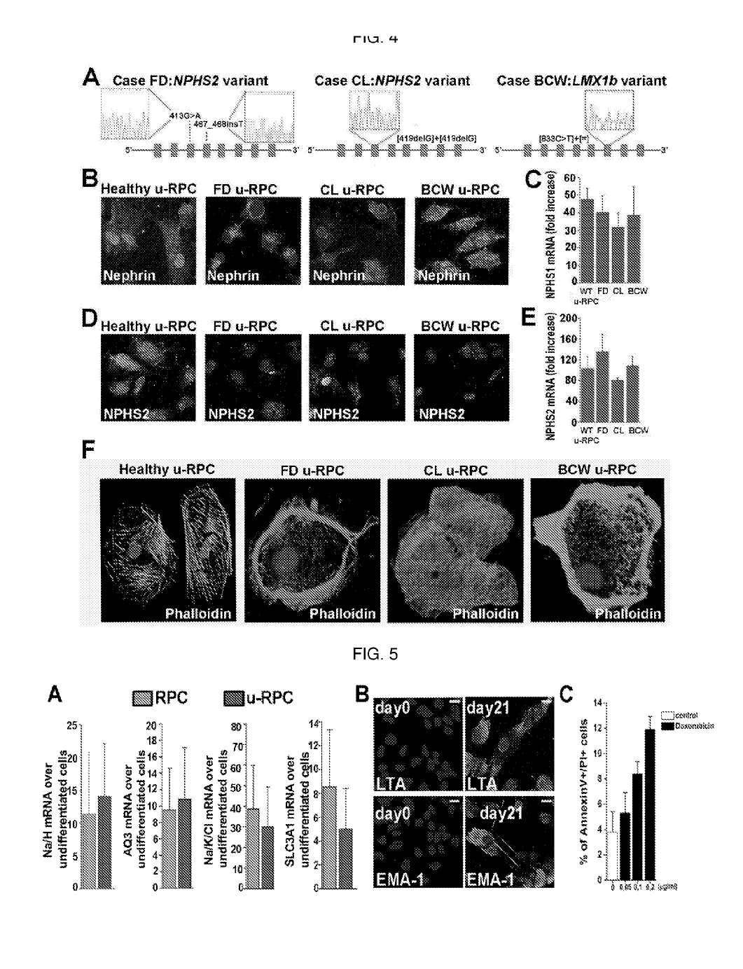

The renal progenitors CD133+CD24+, obtained with the method of the invention, as described above, can therefore be differentiated to the tubular phenotype. Such a differentiation is achieved by the use of the differentiation medium REGM supplemented with HGF (50 ng/mL) for about three weeks. The differentiation of renal progenitors to the tubular phenotype results in an increase of the expression of mRNA levels of a series of markers characteristic of different portions of the tubule, such as the channels Na/H exchanger (Na/H), Aquaporin 3 (AQ3), Na/K/Cl transporter (Na/K/Cl) and amino acid transporter (SLC3A1) (FIG. 5A). In addition, the renal progenitors differentiated to tubule acquire the property of bonding lectinaTetragonolobus (LTA) and of expressing the tubular marker Epithelial Membrane Antigen-1 (EMA-1) (FIG. 5B) at the protein level, respectively characteristic of the proximal and distal convoluted tubule.

After checking the differentiation, the tubular cells are exposed to increasing doses of potentially nephrotoxic drugs such as, for example, doxorubicin, in order to demonstrate that the cell model is able to perfectly mimic the harmful effect of the drug as observed in the clinic. The toxicity of the drug is determined by evaluating the percentage of dead cells after 24 hours of exposure to the potentially nephrotoxic compound, by means of flow cytometry with Annexin V and propidium iodide (PI) (FIG. 5C). The cultures of renal progenitors may, therefore, be used as an in vitro cellular model predictive of a possible cellular toxicity, highlighting the sensitivity of the individual patient to the type and dosage of the drug treatment. The above method allows a more careful selection of the chemotherapy drug to be used for the patient and also allows determining the appropriate dosage to determine the desired therapeutic effect, limiting the long-term side effect of toxicity on the kidneys.

A further object of the present invention is a kit of parts for the simultaneous, separate or sequential use in the method of the invention, said kit comprising at least one container containing a culture medium comprising EGM-MV 20% FBS and a mixture of antibiotics including penicillin, streptomycin and rifampicin.

In said kit, preferably, the culture medium consists of EGM-MV 20% FBS and a mixture of antibiotics consisting of penicillin, streptomycin and rifampicin. More preferably, said mixture of antibiotics consists of 100 U/mL penicillin, 1 mg/mL streptomycin and 8 mcg/mL rifampicin.

Said kit preferably further comprising at least one container containing antibodies anti-CD133 and at least one container containing antibodies anti-CD24 and optionally at least one container containing antibodies anti-CD106. The kit according to the invention, preferably, further comprises at least one container comprising differentiation medium (VRAD) consisting of DMEM/F12 supplemented with vitamin D3 and retinoic acid, for the differentiation to podocyte phenotype, or at least one container of differentiation medium REGM supplemented with HGF (50 ng/mL), for the differentiation to tubular phenotype.

The present invention will be better understood in the light of the following embodiments.

### EXPERIMENTAL PART

### Example 1

### Urine Samples

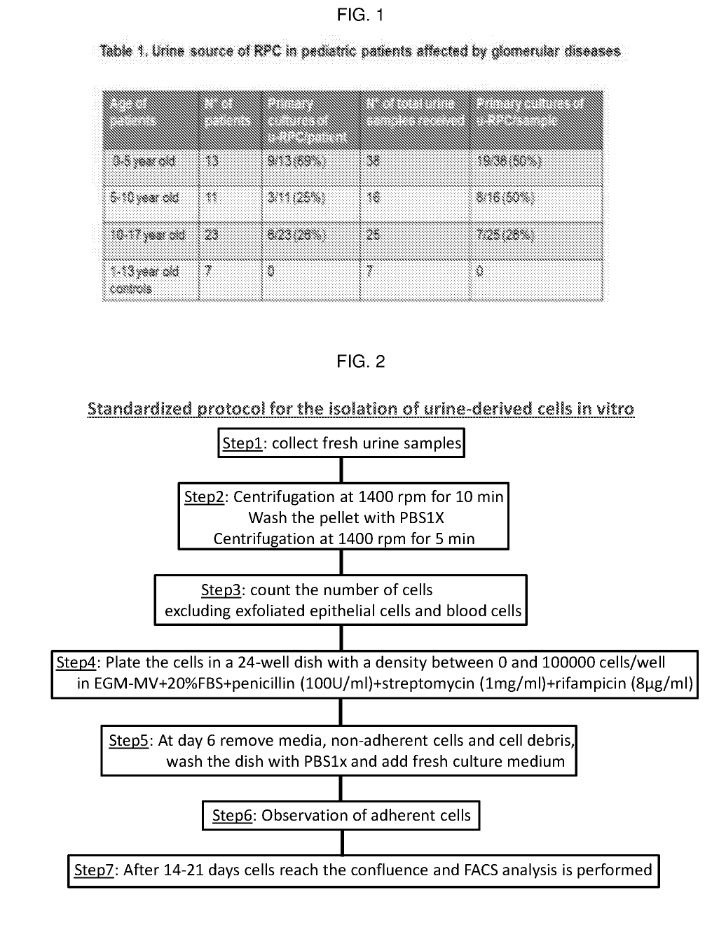

A total of 79 urine samples were collected from 47 pediatric patients aged between 0 and 17 years old and suffering from various glomerular diseases. As a control, urines were collected from healthy children aged between 1 and 13 years (Table 1 in FIG. 1).

### Example 2

### Isolation, Purification and Amplification of Renal Progenitors

The urine samples were centrifuged at 1500 rpm for 10 min, once the supernatant was removed, they were subjected to a second centrifuge in PBS 1× at 1500 rpm for 5 min (see flowchart in FIG. 2). Finally, after removing the supernatant, the cells were re-suspended in EGM-MV 20% FBS. Most of the cells in the urine did not attach to the culture plate and were eliminated at the change of the medium after 6 days of culture. Following plating, only a few cells give rise to a compact and uniform cluster. Since bacterial contamination is a frequent phenomenon in this type of samples, all the cultures obtained were evaluated in PCR for the presence of the bacterial 16S ribosomal RNA gene. Subsequently, the PCR product was subjected to sequencing and comparison of the sequence of the 16S ribosomal RNA gene with those present in Genbank showed a homology of 95% with bacteria belonging to the group of Enterococci. A specific mixture of antibiotics consisting of penicillin (100 U/mL), streptomycin (1 mg/mL) and rifampicin (8 μg/mL) was added to all cultures obtained and a further analysis for the presence of the bacterial ribosomal RNA was repeated after two weeks of treatment. At the end of this period, if the cells were free of bacterial contamination, the antibiotics were removed. This treatment was included in a standardized protocol, according to the invention, for the preparation of cell cultures from urine samples as shown in FIG. 2. This method of isolation of cells from urine allowed obtaining primary cultures with high efficiency and reproducibility (18 patients out of 47 patients, 38.3%), and in particular cell cultures were obtained from 13 patients with exponential growth rate which allowed expanding them indefinitely. No control patient gave rise to cell cultures as shown in Table I (FIG. 1).

### Example 3

### Characterization of Renal Progenitors Isolated from Urine

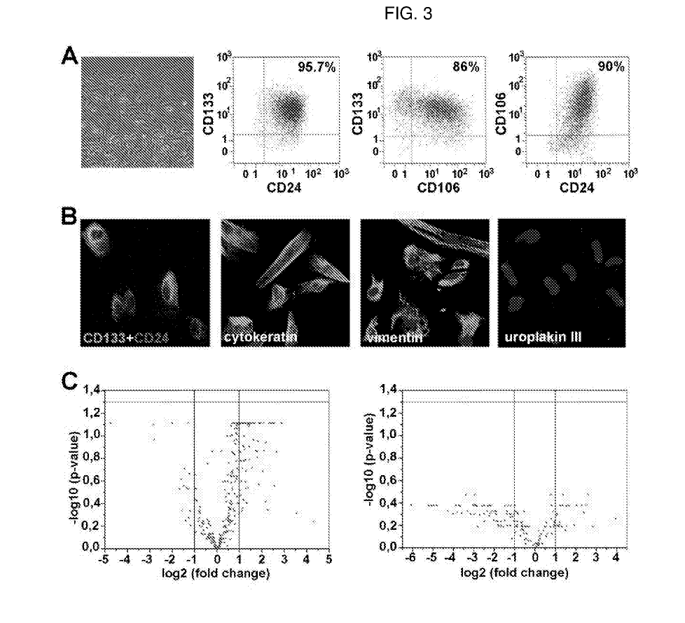

Cells isolated from the urine, according to the method of the invention, show a morphology similar to that of the renal progenitors CD133+CD24+ isolated from kidney tissue (FIG. 3A), and express with high intensity the surface markers characteristic of renal progenitors such as CD133, CD24 and CD106, as demonstrated after flow cytometric analysis, usually in percentages higher than 90% (FIG. 3A). These results show that the isolation method according to the present invention allows obtaining, with high purity, an extremely homogeneous population of renal progenitors CD133+CD24+. Furthermore, after confocal microscopy analysis, the cells isolated from the urine also showed to be homogeneous for the expression of the markers cytokeratin and vimentin (FIG. 3B), while they do not express uroplakin III, a marker characteristic of urothelium, demonstrating, therefore, the renal origin of the cells isolated from the urine (FIG. 3B). In addition, the cells were also assessed for their gene expression profile of the G-proteincoupledreceptor (GPCRs) (380 genes), of the genes characteristic of inflammation (92 genes) and of microRNA (168 genes), revealing that the gene profile of cells isolated from the urine, according to the method of the invention, is not significantly different from that of the renal progenitors CD133+CD24+ as shown in FIG. 3C, and therefore it is the same population.

### Example 4

### Differentiation of Renal Progenitors Isolated from the Urine to Podocyte and Study of the Functional Role of Mutations on the Podocin Gene (NPHS2) And On The LMX1B Gene

Patient 1 (case FD) had a compound heterozygous mutation in the NPHS2 gene (NPHS2 c.[413G>A]+[467_468insT]) consisting of a known missense mutation of one allele and an unknown mutation that causes a frameshift on the coding sequence on the other allele. This latter mutation results in the appearance of a STOP codon that leads to the translation of the truncated podocin in the C-terminal portion. Patient 2 (case CL) had a known homozygous mutation in the NPHS2 gene (NPHS2 c.[419deIG]+[419delG]) able to determine a frameshift on the coding sequence that determines the appearance of a STOP codon that leads to the translation of a truncated protein in the C-terminal portion. Patient 3 (case BCW) had an unknown heterozygous missense mutation in the LMX1B gene (LMX1B c.[833C>T]+[=]) (FIG. 4A). In order to study the functional role of these mutations on the podocyte, the renal progenitors CD133+CD24+ obtained by the method of the invention from the urine of the three patients were differentiated to podocyte and subsequently assessed for the acquisition of the expression of the nephrin and podocin proteins after differentiation. In order to differentiate the renal progenitors CD133+CD24+ to the podocyte phenotype, the cells were cultured in the differentiation medium (VRAD) consisting of DMEM/F12 supplemented with vitamin D3 and retinoic acid, for 48 h. In all three patients, the expression of nephrin and podocin were assessed (FIG. 4B, D). While nephrin was expressed at normal levels, the expression of podocin was greatly reduced in patients with mutations in the NPHS2 gene, or only moderately reduced in patients with mutation on the LMX1B gene, compared to the expression observed in renal progenitors CD133+CD24+ obtained from pediatric patients without genetic mutation and differentiated to podocyte (FIG. 4B, D). In contrast to what observed on the expression of proteins, no statistically significant variation of mRNA levels of nephrin and podocin was appreciated in the three patients with mutations in the NPHS2 and LMX1B gene compared with the renal progenitors CD133+CD24+ obtained from pediatric patients without genetic mutation (FIG. 4C, E). Given the key role played by podocin and LMX1B in maintaining the proper organization of the cytoskeletal filaments, it was assessed whether the reduced expression of podocin could alter the cytoskeletal architecture of podocytes. In all three patients, the analysis of the cytoskeleton assessed by staining with phalloidin showed that the proper organization of the actin filaments of the cytoskeleton was severely impaired and the number of actin filaments significantly reduced, demonstrating the key role played by podocin and by the transcription factor LMX1B in maintaining the correct architecture of the cytoskeleton in podocytes (FIG. 4F).

### Example 5

### Differentiation of Renal Progenitors Isolated from the Urine to Tubules and Study of the Toxicity of Chemotherapeutic Drugs on Them

The renal progenitors CD133+CD24+, obtained with the method of the invention, as described above, can then be differentiated to the tubular phenotype. In particular, urine is collected from patients who will have to undergo therapy with potentially nephrotoxic drugs and the renal progenitors are isolated. The cultures of patient-specific renal progenitors are differentiated in tubular cells by keeping them in differentiation medium REGM supplemented with HGF (50 ng/mL) for about three weeks. The differentiation of the renal progenitors to the tubular phenotype resulting in the acquisition of markers of differentiated tubular cells (FIG. 5A, B). After checking the differentiation, the tubular cells are exposed to increasing doses of potentially nephrotoxic drugs such as, for example, doxorubicin, in order to demonstrate that the cell model is able to perfectly mimic the harmful effect of the drug as observed in the clinic. The toxicity of the drug is determined by evaluating the percentage of dead cells after 24 hours of exposure to the potentially nephrotoxic compound, by means of flow cytometry with Annexin V and propidium iodide (PI) (FIG. 5C). The cultures of renal progenitors may, therefore, be used as a model predictive of a possible cellular toxicity, highlighting the sensitivity of the individual patient to the type and dosage of the drug treatment. The above method allows a more careful selection of the potentially nephrotoxic drug to be used for the patient and also allows determining the appropriate dosage to determine the desired therapeutic effect, limiting the long-term side effect of toxicity on the kidneys.

1. A method for the isolation, purification and amplification of renal progenitor cells CD133+CD24+ of a patient, said method comprising the following sequence of operations:
subjecting a sample of urine obtained from the patient to a first centrifugation;
removing the supernatant and re-suspending the pellets in PBS;
subjecting to a second centrifugation;
removing the supernatant;
transferring the cells on a cell culture plate and growing in a culture medium comprising EGM-MV 20% FBS and a mixture of antibiotics comprising penicillin, streptomycin and rifampicin;
after 5-7 days of culture, removing the culture medium, washing the culture plate with PBS and then adding said fresh culture medium;
growing for at least another 7-9 days, in said fresh culture medium and then subsequently up to confluence of a population of cells characterized by the expression of surface markers CD133 and CD24 characteristic of renal progenitors.

2. A method according to claim 1, wherein said mixture consists of penicillin, streptomycin and rifampicin.

3. A method according to claim 2, wherein said mixture consists of 100 U/mL penicillin, 1 mg/mL streptomycin and 8 mcg/mL rifampicin.

4. A method according to claim 1, wherein after the first 12-16 days of total culture in culture medium with a mixture of antibiotics, the culture is kept in the absence of antibiotics.

5. A method according to claim 1, wherein the patient suffers from a renal disease or from any disease which requires subjecting to treatment with potentially nephrotoxic drugs.

6. A method according to claim 5, wherein said glomerular renal disease is genetic.

7. A diagnostic method for renal diseases, said diagnostic method comprising the method of isolation of renal progenitors according to claim 1.

8. Renal progenitor cells isolated from the urine of a patient by the method according to claim 1, said cells expressing CD133 and CD24 characteristic of renal progenitors.

9. A method comprising the use of cells according to claim 8, differentiated to podocyte or tubular phenotype as in vitro cellular models for the screening of drugs for the treatment of renal diseases or for the patient-specific prediction of renal toxicity of drugs.

10. A method comprising the use of the cells according to claim 8, differentiated to podocyte or tubular phenotype as cellular models for the in vitro study of the functional role of unknown mutations involved in renal diseases.

11. A kit of parts for the simultaneous, separate or sequential use in the method according to claim 1, said kit comprising at least one container containing a culture medium comprising EGM-MV 20% FBS and a mixture of antibiotics including penicillin, streptomycin and rifampicin.
